(19) United States
(12) Patent Application Publication
(10) Pub. No.: US 2015/0290136 A1
(43) Pub. Date: Oct. 15, 2015

(54) PROCESS FOR PREPARING A GRANULATED PRODUCT FROM A POWDER COMPOSITION

(71) Applicants: Shraddha Sanjeev JOSHI, Navi Mumbai (IN); Ashish Sharadchandra GUHA, (US); Vinay JAIN, (US)

(72) Inventors: Shraddha Sanjeev JOSHI, Navi Mumbai (IN); Ashish Sharadchandra GUHA, Mumbai, Dombivali (E) (IN); Vinay JAIN, Indore (M.P.) (IN)

(73) Assignee: Evonik Roehm GmbH, Darmstadt (DE)

(21) Appl. No.: 14/646,569

(22) PCT Filed: Feb. 28, 2013
(86) PCT No.: PCT/EP13/54060

§ 371 Date: May 21, 2015

(30) Foreign Application Priority Data
Nov. 22, 2012 (IN) 4879/CHE/2012

(51) Int. Cl. A61K 9/16

(52) U.S. Cl. CPC A61K 9/1694; A61K 9/1635; A61K 9/1617

(57) ABSTRACT

The invention discloses a process for preparing granulated product from a powder composition comprising the mixed components (a) and (b) and (c) or (d) or both, with (a), a copolymer consisting of free-radical-polymerized C1- to C4-esters of acrylic or methacrylic acid and alkyl(meth)acrylate monomers with tertiary amino groups in the alkyl side groups (b) 5 to 25% by weight, based on (a), of a C₁₂- to C₁₈-monocarboxylic acid or a C₁₂- to C₁₈-alcohol, (c) 0 to 10% by weight based on (a) of a dicarboxylic acid having 3 to 10 carbon atoms, (d) 0 to 20% by weight, based on (a), of an emulsifier having an HLB of at least 14, wherein the mixed components are processed by a dry granulation process to a granulate with a mean particle size of 2 mm or less.

### FIELD OF THE INVENTION

The inventions discloses a roll compacted granulated composition of an amino(meth)acrylate copolymer with increased storage stability.

### TECHNICAL BACKGROUND

WO 02/67906 describes a process for the production of a coating and binding agent for oral or dermal pharmaceutical forms consisting essentially of

(a) a copolymer consisting of free-radical-polymerized C1- to C4-esters of acrylic or methacrylic acid and further (meth)acrylate monomers which have functional tertiary ammonium groups, the copolymer being present in powder form having an average particle size of 1-40 μm (e.g. EUDRAGIT® E PO)
(b) 3 to 15% by weight, based on (a), of an emulsifier having an HLB of at least 14
(c) 5 to 50% by weight, based on (a), of a C₁₂- to C₁₈-monocarboxylic acid or a C₁₂- to C₁₈-hydroxyl compound,
the components (a), (b) and (c) being blended or mixed with one another with or without addition of water and optionally with addition of a pharmaceutical active compound and further customary additives and the coating and binding agent being produced from the mixture by melting, casting, spreading, spraying or granulating. A typical composition may comprise EUDRAGIT® E PO, sodium laurylsulfate and stearic acid.

WO 2011/012161A1 describes a powdery or granulated composition comprising at least by 30% by weight of a mixture of the components (a), (b) and (c) with

(a) a copolymer composed of polymerized units of C₁- to C₄-alkyl esters of acrylic or methacrylic acid and of alkyl(meth)acrylate monomers with a tertiary amino group in the alkyl radical and
(b) 0.5 to 10% by weight based on (a) of a dicarboxylic acid having 3 to 10 carbon atoms and
(c) 5 to 20% by weight based on (a) of a fatty monocarboxylic acid having 8 to 18 carbon atoms.

The inventive composition is intended to be used as a rapidly in water dissolving powder or granulate. The dispersed aqueous compositions show a low viscosity and can therefore be processed directly as coating and binding agents for pharmaceutically, nutraceutically or cosmetically purposes.

WO 2011/012335A1 describes a powdery or granulated composition comprising at least by 30% by weight of a mixture of

(a) a copolymer composed of polymerized units of C₁- to C₄-alkyl esters of acrylic or methacrylic acid and of alkyl(meth)acrylate monomers with a tertiary amino group in the alkyl radical and
(b) 5 to 15% by weight based on (a) of a salt of a fatty monocarboxylic acid having 10 to 18 carbon atoms, and
(c) 10 to 20% by weight based on (a) of fatty monocarboxylic acid having 8 to 18 carbon atoms and/or a fatty alcohol having 8 to 18 carbon atoms.

The inventive composition is intended to be used as a rapidly in water dissolving powder or granulate. The dispersed aqueous compositions show a low viscosity and can therefore be processed directly as coating and binding agents for pharmaceutically, nutraceutically or cosmetically purposes.

### Problem and Solution

Copolymers consisting of free-radical-polymerized C1- to C4-esters of acrylic or methacrylic acid and alkyl(meth)acrylate monomers with tertiary ammonium groups in the alkyl side groups, the copolymer being present in powder form are well known in the pharmaceutical and the nutraceutical industry for instance under the trade name EUDRAGIT® E PO.

Powdery or granulated compositions of components mentioned in patents WO 02/67906 and WO 2011/012161 A1 suffer from the drawback of agglomeration and lump formation in the mixture of powder on storage stability.

It was an object of the present invention to provide a ready to use product based on a copolymer consisting of free-radical-polymerized C1- to C4-esters of acrylic or methacrylic acid and alkyl(meth)acrylate monomers with tertiary ammonium groups in the alkyl side groups, the copolymer being present in powder form, with improved flow properties and improved storage stability for at least 6 months or more.

The problem was solved according to the features as claimed.

### DETAILED DESCRIPTION OF THE INVENTION

### Definitions

### Mean Particle Size

The mean particle size of the powder components can be determined as follows: By air-jet screening for simple separation of the ground product into a few fractions. In the present measurement range, this method is somewhat less accurate than the alternatives. At least 70%, preferably 90% of the particles relative to the weight (weight distribution), however, should lie within the intended size range.

A highly suitable measuring method for smaller particles, such as SiO₂ particles, is laser refraction for determination of particle size distribution. Commercial instruments permit measurement in air (Malvern Co. S3.01 Particle Sizer) or preferably in liquid media (LOT Co., Galai CIS 1). The prerequisite for measurement in liquids is that the polymer does not dissolve therein or the particles do not change in some other way during the measurement. An example of a suitable medium is a highly diluted (about 0.02%) aqueous Polysorbate 80 solution.

### Angle of Repose

A variety of angle of repose test methods are described in the literature. Those methods are well known to a person skilled in the art. A test method which is suitable for determining the static angle of repose in the sense of this invention can be for instance classified on the basis of the following two important experimental variables:
1. The height of the “funnel” through which the powder passes may be fixed relative to the base, or the height may be varied as the pile forms.
2. The base upon which the pile forms may be of fixed diameter or the diameter of the powder cone may be allowed to vary as the pile forms.

Form the angle of repose on a fixed base with a retaining lip to retain a layer of powder on the base. The base should be free of vibration. Vary the height of the funnel to carefully build up a symmetrical cone of powder. Care should be taken to prevent vibration as the funnel is moved. The funnel height should be maintained approximately 2-4 cm from the top of the powder pile as it is being formed in order to minimize the impact of falling powder on the tip of the cone. If a symmetrical cone of powder cannot be successfully or reproducibly prepared, this method is not appropriate. Determine the angle of repose by measuring the height of the cone of powder and calculating the angle of repose, α, from the following equation:

$$tan ( α ) = \frac{height}{0.5 base}$$

### Powder Composition (Before Roll the Dry Granulation Process)

A powder composition as described in here may consist of particles, preferably spherical particles, having a mean particle size of less than 150, for instance in the range from about 1 to 120, preferably in the range from about 1 to 100 μm. Usually the mean particle sizes of the mixed components and excipients present in the mixture do not differ from each by more than +/−100 or +/−50% to avoid demixing effects.

### Dry Granulation Process

A dry granulation may be defined in that it converts powder particles into granules by using the application of pressure without the intermediate use of a liquid. It therefore avoids conditions that might cause the degradation of the product.

Usually two pieces of equipment are necessary for dry granulation: first, means, for instance a machine, for compressing or compaction of the dry powders into intermediate products, which may be sheets, ribbons or flakes, and secondly means, for instance a mill, for breaking up these intermediate products into granules. Such a granulate may result in granules with a mean particle size of 2 mm or less preferably from 0.15 to 2, from 0.5 to 1.8 or 0.25 to 1.0 mm.

A suitable dry granulation process may be a slugging process or a roll compaction process. Dry granulation processes are well known to a skilled person in the field of pharmacy or galenics.

### Slugging

Slugging is a dry granulation process which may be defined as a pre-compression process for the formation of extra large tablets (slugs). Dry powders may be compressed for instance by using a conventional compression tablet machine or by using a large heavy-duty rotary press. This process is known as ‘slugging’, the compact materials from the process, typically 10-30 mm diameter by about 5-20 mm thick, may be termed a ‘slug’.

The resulting intermediate products, slugs, may be subsequently broken down into granules. A hammer mill or oscillating granulator is suitable for breaking the slugs into granules.

### Roll Compaction

Roll compaction is a dry granulation process wherein a powder composition is squeezed between two rollers to produce a sheet or flakes of materials. A suitable equipment for roll compaction is a roller compactor, which is also commonly referred to as a chilsonator. A powder composition may be converted by means of the roll compaction to flakes or ribbons. These sheets, flakes or ribbons may vary widely in size and shape and may have a length of about more than 2 and up to 100 mm, for instance 5 to 50 mm, a broadness of about 1 to 30 cm and a height or thickness in the range of more than 2 up to 10 mm, for instance of 3 to 5 mm.

### Granulated Product

A granulated product may be obtained by the dry granulation process as described herein. Slugs respectively sheets, flakes or ribbons are obtained from a slugging process or from a roll compaction process step. These intermediate products may be subsequently comminuted by cracking or milling in a mill or by means of other suitable equipment to a granulate or to a granulated product with a mean particle size of 2 mm or less, preferably from 0.15 to 2, from 0.5 to 1.8 or 0.25 to 1.0 mm. The granulated product particles may be preferably spherical or almost spherical or at least of more or less regular spherical. The granulated particles may also be more or less of short cylindrical form or of irregular spherical form. The granulated particles may subsequently be compressed into tablets or may be filled into capsules.

### Emulsifier

An emulsifier may be defined as a molecule or a substance comprising a balance of hydrophilic and hydrophobic (lipophilic) properties. This may also be called an amphiphilic property. Emulsifiers may be characterized by their HLB values (HLB stands for hydrophilic-lipophilic balance)

The HLB, introduced by Griffin in 1950, is a measure of the hydrophilicity of lipophilicity of nonionic surfactants. It may be determined experimentally by the phenol titration method of Marszall; cf. “Parfümerie, Kosmetik”, Volume 60, 1979, pp. 444-448; further literature references are in Römpp, Chemie-Lexikon, 8^{th} ed. 1983, p. 1750. See also, for example, U.S. Pat. No. 4,795,643. An HLB (hydrophilic/lipophilic balance) can be determined exactly only for nonionic emulsifiers. For anionic emulsifiers, this value may be determined arithmetically but is virtually in most cases above or well above 20.

### Process

The invention is concerned with a process for preparing a granulated product from a powder composition comprising the mixed components (a) and (b) and (c) or (d) or both (which means (a), (b) and ((c) or (d)) or (a), (b), (c) and (d)), with

(a), a copolymer consisting of free-radical-polymerized C1- to C4-esters of acrylic or methacrylic acid and alkyl(meth)acrylate monomers with tertiary amino groups in the alkyl side groups
(b) 5 to 25% by weight, based on (a), of a C₁₂- to C₁₈-monocarboxylic acid or a C₁₂- to C₁₈-alcohol,
(c) 0 to 10% by weight based on (a) of a dicarboxylic acid having 3 to 10 carbon atoms,
(d) 0 to 20% by weight, based on (a), of an emulsifier having an HLB of at least 14,
wherein the mixed components are processed by a dry granulation process to a granulate with a mean particle size of 2 mm or less.

Thus the powder composition may comprise or contain the components
(a), (b) and (c) or
(a), (b) and (d) or
(a), (b), (c) and (d).

The mixed components are preferably processed or compacted by the dry granulation process, for instance a slugging or a roll compaction process, at first to intermediate products, for instance slugs, sheets, flakes or ribbons, which are then subsequently comminuted to the final granulate with a mean particle size of 2 mm or less.

### Component (a)

Component (a) is a copolymer consisting of free-radical-polymerized C1- to C4-esters of acrylic or methacrylic acid and alkyl(meth)acrylate monomers with tertiary amino groups in the alkyl side groups.

Preferably component (a) is a copolymer composed of polymerized units of 30 to 80% by weight of C₁- to C₄-alkyl esters of acrylic or of methacrylic acid and 70 to 20% by weight of alkyl(meth)acrylate monomers having a tertiary amino group in the alkyl radical.

Preferably component (a) is a copolymer composed of polymerized units of 20-30% by weight of methyl methacrylate, 20-30% by weight of butyl methacrylate and 60-40% by weight of dimethylaminoethyl methacrylate.

Preferably the component (a) is present in powder form with a mean particle size of less than 150, from 1 to 120, from 1 to 100, from 1 to 40, preferably from 5 to 25 μm.

Component (a) is an amino(meth)acrylate copolymer that may be composed partly or fully of alkyl acrylates and/or alkyl methacrylates having a tertiary amino group in the alkyl radical. Suitable (meth)acrylate copolymers are known, for example, from EP 0 058 765 B1.

Suitable monomers with functional tertiary amino groups are detailed in U.S. Pat. No. 4,705,695, column 3 line 64 to column 4 line 13. Mention should be made in particular of dimethylaminoethyl acrylate, 2-dimethylaminopropyl acrylate, dimethylaminopropyl methacrylate, dimethylaminobenzyl acrylate, di methylaminobenzyl methacrylate, (3-dimethylamino-2,2-dimethyl)propyl acrylate, dimethylamino-2,2-dimethyl)propyl methacrylate, (3-diethylamino-2,2-dimethyl)propyl acrylate and diethylamino-2,2-dimethyl)propyl methacrylate. Particular preference is given to dimethylaminoethyl methacrylate.

A specifically suitable commercial amino (meth)acrylate copolymer is, for example, formed from 25% by weight of methyl methacrylate, 25% by weight of butyl methacrylate and 50% by weight of dimethylaminoethyl methacrylate (EUDRAGIT® E100 or EUDRAGIT® E PO (powder form, with an average particle size of around 15 μm). EUDRAGIT® E100 and EUDRAGIT® E PO are water-soluble below approx. pH 5.0 and are thus also gastric juice-soluble.

Suitable copolymers may be the “amino methacrylate copolymer (USP/NF)”, “basic butylated methacrylate copolymer (Ph. Eur)” or “aminoalkyl Methacrylate Copolymer E (JPE)” which are of the EUDRAGIT® E type.

A further (meth)acrylate copolymer with tertiary amino groups may be, for example, formed from 50-60, preferably 55% by weight of methyl methacrylate and 40-50, preferably 45% by weight of diethylaminoethyl methacrylate (s. WO2009016258, WO2010139654 and WO2012041788A1).

### Component (b)

Component (b) is a C₁₂- to C₁₈-monocarboxylic acid or a C₁₂- to C₁₈-alcohol, comprised or contained in amounts of 5 to 25, preferably 10 to 20% by weight, based on component (a).

Component (b) may be lauric acid, palmitic acid, stearic acid, lauryl alcohol, palmityl alcohol or stearyl alcohol.

Component (b) is a powder product. The mean particle size of component (b) may be less than 150 μm, preferably less than 100 μm, for instance in the range of 10 to 100 μm.

### Component (c)

Component (c) is a dicarboxylic acid having 3 to 10 carbon atoms.

Component (c) is an optional component and may be comprised or contained in amounts of 0 to 10, 0.1 to 8 or preferably 1 to 6% by weight, based on component (a).

Component (c) may be for instance fumaric acid, malic acid, tartaric acid, succinic acid or any mixtures thereof.

Component (c) may be used as a powder product. The mean particle size of component (c) may be less than 150 μm, preferably less than 100 μm, more preferably in the range of 10 to 100 μm.

### Component (d)

Component (d) is an emulsifier having an HLB of at least 14.

Component (d) is an optional component and may be comprised or contained in amounts of 0 to 20, 1 to 18, preferably 5 to 15% by weight, based on component (a).

Component (d) may be for instance sodium lauryl sulfate or polysorbate 80.

Preferred emulsifiers in respect to component (d) are non-ionic or anionic emulsifiers. Further preferred, the emulsifiers in respect to component (b) may be selected from the group consisting of fatty alkyl sulfates, preferably sodium laurylsulfate, sodium cetylstearylsulfate, saccharose stearate, polysorbates, especially polysorbate 80 (Tween® 80) or mixtures thereof.

Component (d) may be used as a powder product. The mean particle size of component (d) may be less than 150 μm, preferably less than 100 μm, for instance in the range of 10 to 100 μm.

### Active Pharmaceutical Ingredients

The powder composition or powder mixture may further comprise or contain one or more active pharmaceutical ingredients.

### Further Excipients

The powder composition or powder mixture may further comprise or contain excipients selected from the groups of antioxidants, brighteners, binding agents, flavouring agents, flow aids, fragrances, glidants, penetration-promoting agents, pigments, plasticizers, polymers, pore-forming agents or stabilizers. The further excipients are different from the components (a) to (d). Further excipients may be added in amounts of 0 to 200% by weight calculated on component (a).

The powder mixture may comprise or contain talc or glycerol monostearate as further excipients. Talc may be added for instance in amounts of 30 to 120, preferably 40 to 80% by weight calculated on component (a) or glycerol monostearate in amounts of 0.1% to 10% calculated on component (a)

### Silicon Dioxide

Preferably silicon dioxide (SiO₂) may be added as a further excipient. Colloidal SiO₂ of the Aerosil® type, which is produced by a flame process and which usually has average particle sizes in the range below 100 nm is preferred. The mean particle size of the SiO₂ may be preferably in the range of 1 to 80 nm. Silicon dioxide may be added in an amount of 0.1 to 10, more preferably 0.5 to 5% by weight calculated on component (a).

### Preferred Powder Compositions or Powder Mixtures

A preferred powder composition or powder mixture may comprise or contain
component (a),
component (b) 5 to 25% by weight stearic acid,
component (d) 5 to 20% by weight sodium lauryl sulfate,
and 0 to 120, preferably 30 to 120% by weight talc,
and 0 to 10, preferably 0.1 to 10, more preferably 0.5 to 5% by weight SiO₂
with each percentage by weight calculated on component (a)
(all components and excipients may add up to 100%)

Another preferred powder composition or powder mixture may comprise or contain
component (a),
component (b) 5 to 25% by weight stearic acid,
component (c) 1 to 10% by weight tartaric acid,
and 0 to 120, preferably 30 to 120% by weight talc,
and 0 to 10, preferably 0.1 to 10, more preferably 0.5 to 5% by weight SiO₂
with each percentage by weight calculated on component (a)
(all components and excipients may add up to 100%)

### Advantages

The disclosed process is especially of advantage for granulated product mixtures comprising or containing substances, such as stearic acid as a component (b). Without being bound to a theory the inventors assume that the dry granulation process, for instance slugging or roll compaction, improves the bulk density of a previous powder mixture which results in improvement in the flow of mixture and also improve the dispersibility of the resulting granulated product mixture in the solvent used for preparation of dispersion. However it was surprisingly observed that storage stability of the granulated product was also improved at ambient temperature and humidity condition after addition of silicon dioxide into the granulated composition. This was by no means to be foreseen and is of practical advantage. Especially for those above mentioned preferred powder mixtures that are in practice widely used, comprising component (a), 5 to 25% by weight stearic acid and 5 to 20% by weight sodium lauryl sulfate or 1 to 10% by weight tartaric acid, (a) and 0 to 120% by weight talc and 0 to 10% by weight SiO₂ calculated on component (a), the process is of advantage.

### Examples

EUDRAGIT® E PO is a copolymer composed of 25% by weight of methyl methacrylate, 25% by weight of butyl methacrylate and 50% by weight of dimethylaminoethyl methacrylate in powder form, with an average particle size of around 15 μm.

### Excipients

All excipients were used in pharmaceutical quality

Aerosil® 200 is a SiO₂ powder with a mean particle size of about 17 nm

### Storage Stability

For testing storage stability powders or granulates were stored at 25° C./60% relative humidity (RH) in HDPE containers.

Accelerated Stability was tested at 40° C./75% RH in open Petri dishes

### Classification of the Storage Stabilities

After storage, physical appearance and flow behaviour of the powders or granulates were classified as follows:

| | | |
|---|---|---|
| +++ | Exellent | Free flowing, No agglomeration |
| ++ | Good | Flowable, No agglomeration |
| + | Acceptable | Poor Flow, No agglomeration |
| − | Bad | Few agglomerates/Lumps |
| −− | Very Bad | Agglomerates/Lager lumps, Sticking |
| −−− | Severe | Severe Sticking, Sticky mass |

### Procedure for Angle of Repose:

The angle of repose (unit degrees, [°]) was classified on the basis of the following two experimental variables:
1. The height of the “funnel” through which the powder passes may be fixed relative to the base, or the height may be varied as the pile forms.
2. The base upon which the pile forms may be of fixed diameter or the diameter of the powder cone may be allowed to vary as the pile forms.

Form the angle of repose on a fixed base with a retaining lip to retain a layer of powder on the base. The base should be free of vibration. Vary the height of the funnel to carefully build up a symmetrical cone of powder. Care should be taken to prevent vibration as the funnel is moved. The funnel height should be maintained approximately 2-4 cm from the top of the powder pile as it is being formed in order to minimize the impact of falling powder on the tip of the cone. If a symmetrical cone of powder cannot be successfully or reproducibly prepared, this method is not appropriate. Determine the angle of repose by measuring the height of the cone of powder and calculating the angle of repose, a, from the following equation:

$$tan ( α ) = \frac{height}{0.5 base}$$

TABLE 1
Storage stabilities of an EUDRAGIT ® E PO formulation comprising Stearic acid and SLS;.

| | | | At 25 C./60% RH stored in | | | |
|---|---|---|---|---|---|---|
| | | | HDPE container | | | |
| | | with or without | | 1 | 3 | 6 |
| | Description | Aerosil 200 | Initial | Month | Months | Months |
| 1 | Powder composition: EUDRAGIT ® E PO + SLC (10% on | | | | | |
| | EPO) + Stearic acid (15% on EPO) + Talc (50% on EPO) | | | | | |
| 1 (a) | Powder composition (Before roll compaction) | without Aerosil ® 200 | + | − | −− | −− |
| 1 (b) | Powder composition (Before roll compaction) | with Aerosil 200 | + | − | −− | −− |
| 2 | Roll Compacted Granules containing EUDRAGIT EPO + | | | | | |
| | SLS (10% on EPO) + Stearic acid (15% on EPO) + Talc | | | | | |
| | (50% on EPO) | | | | | |
| | Particle size range of granules | | | | | |
| 2 (a) | All particle size granules including fines (<150 microns) | without Aerosil ® 200 | +++ | − | − | − |
| 2 (b) | All particle size granules including fines (<150 microns) | with Aerosil ® 200 | +++ | +++ | +++ | +++ |
| 2 (c) | 150 microns to 850 microns | without Aerosil ® 200 | +++ | − | − | − |
| 2 (d) | 150 microns to 850 microns | with Aerosil ® 200 | +++ | +++ | +++ | +++ |
| 2 (e) | 425 microns to 850 microns | without Aerosil ® 200 | +++ | − | − | − |
| 2 (f) | 425 microns to 850 microns | with Aerosil ® 200 | +++ | +++ | +++ | +++ |
| 2 (g) | 250 microns to 300 microns | without Aerosil ® 200 | +++ | − | −− | −− |
| 2 (h) | 250 microns to 300 microns | with Aerosil ® 200 | +++ | +++ | +++ | +++ |

SLS = Sodium lauryl sulfate 3.5% by weight Aerosil ® 200 calculated on component (a) was added to granulated product wherever formulation is mentioned with Aerosil ® 200

TABLE 2
Flow Properties of an EUDRAGIT ® E PO formulation comprising stearic acid and SLS;. SLS = Sodium lauryl sulfate

| | | | | | Flow |
|---|---|---|---|---|---|
| | | | | | properties |
| | | | Angle of | | corresponding |
| | | with or without | repose [°] | | to angle of |
| | Description | Aerosil ® 200 | Mean | SD | repose as per USP |
| 1 | Powder composition of Standard EPO formulation: EUDRAGIT | without Aerosil ® 200 | 46.73 | 1.29 | Poor |
| | EPO + SLS (10% on EPO) + Stearic acid (15% on EPO) + Talc | | | | |
| | (50% on EPO) (Before roll compaction) | | | | |
| 2 | Roll Compacted Granules containing EUDRAGIT EPO + | | | | |
| | SLS (10% on EPO) + Stearic acid (15% on EPO) + Talc (50% on EPO) | | | | |
| | Particle size range of granules | | | | |
| 2(a) | All particle size granules including fines (<150 microns) | without Aerosil ® 200 | 39.60 | 0.44 | Fair |
| 2(b) | All particle size granules including fines (<150 microns) | with Aerosil ® 200 | 37.85 | 0.56 | Fair |
| 2(c) | 150 microns to 850 microns | without Aerosil ® 200 | 33.67 | 0.15 | Good |
| 2(d) | 150 microns to 850 microns | with Aerosil ® 200 | 31.95 | 0.29 | Good |
| 2(e) | 425 microns to 850 microns | without Aerosil ® 200 | 31.70 | 0.45 | Good |
| 2(f) | 425 microns to 850 microns | with Aerosil ® 200 | 30.04 | 0.25 | Excellent |
| 2(g) | 250 microns to 300 microns | without Aerosil ® 200 | 34.63 | 1.23 | Good |
| 2(h) | 250 microns to 300 microns | with Aerosil ® 200 | 34.93 | 0.69 | Good |

3.5% by weight Aerosil ® 200 calculated on component (a) was added to granulated product wherever formulation is mentioned with Aerosil ® 200

TABLE 3
Accelerated storage stabilities of an EUDRAGIT ® E PO formulation comprising stearic acid and SLS;.

| | | Accelerated Stability at 40 C./75% RH in open Petri-dishes | | | | | | | |
|---|---|---|---|---|---|---|---|---|---|
| | | | 1 | 8 | 24 | 4 | 7 | 15 | 30 |
| | Description | Initial | Hour | Hours | Hours | Days | Days | Days | Days |
| 1 | Powder composition: EUDRAGIT EPO + SLS | + | −− | −− | −−− | −−− | −−− | −−− | −−− |
| | (10% on EPO) + Stearic acid (15% on EPO) + | | | | | | | | |
| | Talc (50% on EPO) | | | | | | | | |
| 2 | Roll Compacted Granules containing EUDRAGIT | | | | | | | | |
| | EPO + SLS (10% on EPO) + Stearic acid | | | | | | | | |
| | (15% on EPO) + Talc (50% on EPO) | | | | | | | | |
| | Particle size range of granules | | | | | | | | |
| 2 (a) | All particle size granules including fines | ++ | ++ | ++ | ++ | ++ | + | + | − |
| | (<150 microns) with Aerosil ® 200 | | | | | | | | |
| 2 (b) | 150 microns to 850 microns without Aerosil ® 200 | +++ | − | −− | −− | −− | −− | −− | −− |
| 2 (c) | 150 microns to 850 microns with Aerosil ® 200 | +++ | +++ | +++ | +++ | +++ | +++ | +++ | +++ |

SLS = Sodium lauryl sulfate 3.5% by weight Aerosil ® 200 calculated on component (a) was added to granulated product wherever formulation is mentioned with Aerosil ® 200

TABLE 4
Flow Properties of an EUDRAGIT ® E PO formulation comprising stearic acid and tartaric acid

| | | | | | Flow |
|---|---|---|---|---|---|
| | | | | | properties |
| | | | Angle of | | corresponding |
| | | with or without | repose [°] | | to angle of |
| | Description | Aerosil ® 200 | Mean | SD | repose as per USP |
| 1 | Powder composition (before roll compaction): EUDRAGIT EPO + | without Aerosil ® 200 | 47.1633 | 0.63 | Poor |
| | Tartaric acid(3% on EPO) + Stearic acid(15% on EPO) + | | | | |
| | Talc (50% on EPO) | | | | |
| 2 | Roll Compacted Granules containing EUDRAGIT EPO + | | | | |
| | Tartaric acid(3% on EPO) + Stearic acid(15% on EPO) + | | | | |
| | Talc (50% on EPO) | | | | |
| | Particle size range of granules | | | | |
| 2 (a) | All particle size granules including fines (<150 microns) | without Aerosil ® 200 | 37.39 | 0.60 | Fair |
| 2 (b) | All particle size granules including fines (<150 microns) | with Aerosil ® 200 | 35.65 | 0.61 | Good |
| 2 (c) | 150 microns to 850 microns | without Aerosil ® 200 | 36.67 | 0.52 | Fair |
| 2 (d) | 150 microns to 850 microns | with Aerosil ® 200 | 32.16 | 1.54 | Good |
| 2 (e) | 425 microns to 850 microns | without Aerosil 200 | 36.99 | 0.98 | Fair |
| 2 (f) | 425 microns to 850 microns | with Aerosil ® 200 | 33.01 | 0.29 | Good |
| 2 (g) | 250 microns to 300 microns | without Aerosil ® 200 | 33.43 | 0.55 | Good |
| 2 (h) | 250 microns to 300 microns | with Aerosil ® 200 | 32.79 | 0.89 | Good |

3.36% by weight Aerosil ® 200 calculated on component (a) was added to granulated product wherever formulation is mentioned with Aerosil ® 200

TABLE 5
Storage stability of an EUDRAGIT ® E PO formulation comprising stearic acid and tartaric acid

| | | | At 25 C./60% RH stored in HDPE container | | | |
|---|---|---|---|---|---|---|
| | | with or without | | 1 | 3 | 6 |
| | Description | Aerosil ® 200 | Initial | Month | Months | Months |
| 1 | Powder composition (before roll compaction): | | | | | |
| | EUDRAGIT EPO + Tartaric acid (3% on EPO) + | | | | | |
| | Stearic acid (15% on EPO) + Talc (50% on EPO) | | | | | |
| 1 (a) | Powder composition | without Aerosil ® 200 | + | −− | −−− | −−− |
| 1 (b) | Powder composition | with Aerosil ® 200 | + | −− | −−− | −−− |
| 2 | Roll Compacted Granules containing EUDRAGIT | | | | | |
| | EPO + Tartaric acid (3% on EPO) + Stearic acid | | | | | |
| | (15% on EPO) + Talc (50% on EPO) | | | | | |
| | Particle size range of granules | | | | | |
| 2 (a) | All particle size granules including fines | without Aerosil ® 200 | +++ | −− | −−− | −−− |
| | (<150 microns) | | | | | |
| 2 (b) | All particle size granules including fines | with Aerosil ® 200 | +++ | +++ | +++ | +++ |
| | (<150 microns) | | | | | |
| 2 (c) | 150 microns to 850 microns | without Aerosil ® 200 | +++ | −− | −−− | −−− |
| 2 (d) | 150 microns to 850 microns | with Aerosil ® 200 | +++ | +++ | +++ | +++ |
| 2 (e) | 425 microns to 850 microns | without Aerosil ® 200 | +++ | −− | −−− | −−− |
| 2 (f) | 425 microns to 850 microns | with Aerosil ® 200 | +++ | +++ | +++ | +++ |
| 2 (g) | 250 microns to 300 microns | without Aerosil ® 200 | +++ | −− | −−− | −−− |
| 2 (h) | 250 microns to 300 microns | with Aerosil ® 200 | +++ | +++ | +++ | +++ |

3.36% by weight Aerosil ® 200 calculated on component (a) was added to granulated product wherever formulation is mentioned with Aerosil ® 200

TABLE 6
Accelerated storage stability of an EUDRAGIT ® E PO formulation comprising stearic acid and tartaric acid

| | | Accelerated Stability at 40 C./75% RH in open Petri dishes | | | | | | | |
|---|---|---|---|---|---|---|---|---|---|
| | | | 1 | 8 | 24 | 36 | 2 | 3 | 4 |
| | Description | Initial | Hour | Hours | Hours | Hours | Days | Days | Days |
| 1 | Powder composition (before roll compaction): | + | −− | −−− | −−− | −−− | −−− | −−− | −−− |
| | EUDRAGIT EPO + Tartaric acid (3% on EPO) + | | | | | | | | |
| | Stearic acid (15% on EPO) + Talc (50% on EPO) | | | | | | | | |
| 2 | Roll Compacted Granules containing EUDRAGIT | | | | | | | | |
| | EPO + Tartaric acid (3% on EPO) + Stearic acid | | | | | | | | |
| | (15% on EPO) + Talc (50% on EPO) | | | | | | | | |
| | Particle size range of granules | | | | | | | | |
| 2 (a) | All particle size granules including fines | ++ | ++ | ++ | ++ | − | − | −−− | −−− |
| | (<150 microns) with Aerosil ® 200 | | | | | | | | |
| 2 (b) | 150 microns to 850 microns without Aerosil ® 200 | +++ | − | −−− | −−− | −−− | −−− | −−− | −−− |
| 2 (c) | 150 microns to 850 microns with Aerosil ® 200 | +++ | +++ | +++ | +++ | ++ | + | − | −−− |

3.36% by weight Aerosil ® 200 calculated on component (a) was added to granulated product wherever formulation is mentioned with Aerosil ® 200

1. A process for preparing a granulated product from a powder composition, the process comprising:
granulating the powder composition to granulates with a mean particle size of 2 mm or less by dry granulation,
wherein the powder composition comprises components (a), (b), and at least one of (c) and (d):
(a) a copolymer of free-radical-polymerized C1- to C4-esters of acrylic or methacrylic acid and alkyl(meth)acrylate monomers with at least one tertiary amino group in at least one alkyl side group,
(b) 5 to 25% by weight, based on (a), of a C₁₂- to C₁₈-monocarboxylic acid or a C₁₂- to C₁₈-alcohol,
(c) 0 to 10% by weight, based on (a), of a dicarboxylic acid comprising 3 to 10 carbon atoms,
(d) 0 to 20% by weight, based on (a), of an emulsifier having an HLB of at least 14.

2. The process according to claim 1, wherein the dry granulation is a slugging process or a roll compaction process.

3. The process according to claim 1, wherein the component (a) is a copolymer of polymerized units of 30 to 80% by weight of C₁- to C₄-alkyl esters of acrylic or of methacrylic acid and 70 to 20% by weight of alkyl(meth)acrylate monomers with a tertiary amino group in the at least one alkyl side group.

4. The process according to claim 1, wherein the component (a) is a copolymer of polymerized units of 20-30% by weight of methyl methacrylate, 20-30% by weight of butyl methacrylate and 60-40% by weight of dimethylaminoethyl methacrylate.

5. The process according to claim 1, wherein the component (a) is present in a powder form with an average particle size of 1-40 μm.

6. The process according to claim 1, wherein the component (b) is lauric acid, palmitic acid, stearic acid, lauryl alcohol, palmityl alcohol or stearyl alcohol.

7. The process according to claim 1, wherein the component (c) is fumaric acid, malic acid, tartaric acid, succinic acid or any mixture thereof.

8. The process according to claim 1, wherein the component (d) is sodium lauryl sulfate or polysorbate 80.

9. The process according to claim 1, wherein the powder composition comprises one or more active pharmaceutical ingredients or excipients selected from the group consisting of an antioxidant, a brightener, a binding agent, a flavouring agent, a flow aid, a fragrance, a glidant, a penetration-promoting agent, a pigment, a plasticizer, a polymer, a pore-forming agent, and a stabilizer.

10. The process according to claim 1, wherein the powder composition further comprises talc or glycerol monostearate as an excipient.

11. The process according to claim 1, wherein the powder composition comprises SiO₂ as a further excipient.

12. The process according to claim 11, wherein an amount of SiO₂ ranges from 0.1 to 10% by weight based on the component (a).

13. The process according to claim 1, comprising:
granulating the powder composition to roll compacted granulates with the mean particle size of 2 mm by roll compaction, and
removing a fine fraction of less than 150 microns.

14. The process according to claim 1, wherein the powder composition comprises
the component (a),
5 to 25% by weight of stearic acid as the component (b),
5 to 20% by weight of sodium lauryl sulfate as the component (d),
0 to 120% by weight talc, and
0 to 10% by weight SiO₂,
wherein each percentage by weight is calculated based on the component (a).

15. The process according to claim 1, wherein the powder composition comprises
the component (a),
5 to 25% by weight stearic acid as the component (b),
1 to 10% by weight tartaric acid as the component (c),
0 to 120% by weight talc, and
0 to 10% by weight SiO₂,
wherein each percentage by weight is calculated based on the component (a).

16. A granulated product obtained by the process according to claim 1.
